(19) United States
(12) Patent Application Publication
(10) Pub. No.: US 2013/0058923 A1
(43) Pub. Date: Mar. 7, 2013

(54) BIOMOLECULAR INTERACTIONS AND INTERACTION PRODUCTS AS BIOMARKERS FOR DETECTION, DIAGNOSIS, PROGNOSIS AND PREDICTING THERAPEUTIC RESPONSES OF HUMAN DISEASES

(71) Applicant: Qun Huo, Orlando, FL (US)

(72) Inventor: Qun Huo, Orlando, FL (US)

(21) Appl. No.: 13/566,195

(22) Filed: Aug. 3, 2012

Related U.S. Application Data
(60) Provisional application No. 61/514,744, filed on Aug. 3, 2011.

(51) Int. Cl. G01N 33/574; G01N 21/82; C12Q 1/68; G01N 21/64; G01N 27/72; G01N 30/00; A61K 39/395; G01N 33/573; G01N 27/62; B82Y 15/00

(52) U.S. Cl. 424/133.1; 435/7.4; 435/7.23; 436/501; 435/6.14; 424/130.1; 977/773; 977/920

(57) ABSTRACT

Included in the disclosure is description of a method of determining a presence, and/or aggressiveness level, and/or response to therapy of a human disease in a subject. The method involves determining size of nanoparticles upon being exposed to a biological sample or a component of biological sample or pretreated biological sample from the subject to form an assay solution, wherein the average particle size of the assay solution is correlative to the presence, and/or aggressiveness level, and/or response to therapy of disease in said subject

### CROSS-REFERENCE TO RELATED APPLICATIONS

This application is related to U.S. Provisional Application 61/514,744 filed Aug. 3, 2011 to which priority is claimed under 35 USC 119(e). This application is incorporated herein in its entirety.

### BACKGROUND

Biomarkers are molecules, biological species or biological events that can be used to detect, diagnose, prognosis, and predict therapeutic response of a disease. Biomarkers can be proteins, DNAs, RNAs, small molecules, carbohydrates, intact cells, and others. Most research has been focused on measuring the concentration change of biomarkers in biological sample associated with a disease. A biomarker may exist at extremely low concentrations, particularly, in early stage cancer. Accurate determination of low concentration biomarkers has remained as a significant challenge.

Enzymes are a subgroup of biomolecules that are responsible for most of the biochemical reactions that occur in biological systems. As a catalyst, enzyme can exist at very low concentrations, yet, present enormous catalytic effect. Enzymes such as horseradish peroxidase are often used to amplify biomolecular sensing signals. The up-regulation and down-regulation of enzymes are associated with many human diseases. For example, kallikrein proteins are a family of serine protease capable of cleaving peptide bonds of proteins. It is known that many hormone-related cancers, such as prostate, ovarian and breast cancer have elevated kallikreins. Serine proteases can catalyze the breakdown of many proteins, including immunoglobulins. Matrix metalloproteinase (MMPs) are another class of protease which catalyze the breakdown of extracellular matrix, promoting tumor progression and metastasis.

Cancer immunology is a research that studies the interactions between tumor cells and the immune system, the white blood cells. Enormous evidence from cancer immunology research has shown that at early stage of cancer development, tumor cells and tissues are recognized by the immune system as “non-self”, therefore, a defensive response is triggered by the white blood cells to halt tumor growth and progression. As the immune system works by releasing specific antibody, IgG molecules to bind with tumor-shed antigens, the tumor antigens (TAs) should mostly exist as immunocomplexes, not as individual proteins. Furthermore, IgG-TA complexes will bind with various Fc receptors, and such complex formation prepares the immunocomplexes to be eliminated from the system.

In addition to immunocomplexes, human blood contains a large number of abundant proteins such as serum albumin, α-antitrypsin, α2-macroglobulin, etc. The tumor-shed proteins, if they are not complexed with IgG, may bind with other abundant serum proteins in the blood. A good example is the presence of different forms of PSA, free PSA versus total PSA. PSA forms a complex with a-antichymotrypsin (ACT) in the blood. A typical molar ratio of PSA-ACT complex versus free PSA in most prostate cancer patients was found to be 90:10. In recognition of this fact, the WHO (World Health Organization) established a standard PSA material for diagnostic testing according to this composition. This situation happens to PSA, and most likely could happen to other cancer biomarker proteins as well.

Prostate cancer (PCa) is the most common malignancy and the third leading cause of cancer death in American men. Using digital rectal examination (DRE) combined with PSA (prostate specific antigen) test, most prostate cancer cases are now detected at early stage. However, PSA test cannot distinguish aggressive, metastasizable prostate cancer from latent tumor. According to statistics, “30% of tumors removed by radical prostatectomy are deemed clinically insignificant and would not have required such invasive treatment”. Over-diagnosis and treatment of low-risk prostate cancer has serious and long-lasting side effect: as high as 70% of the patients who receive radical prostatectomy treatment will suffer erectile dysfunction that cannot be remedied by drugs such as Viagra. On the other hand, misdiagnosis of high-risk malignant prostate cancer increases the level of difficulty in treatment and decreases the survival rate of the cancer patients. There is a pressing need to develop new biomarkers and tests that can clearly distinguish aggressive prostate cancer from normal, non-cancerous benign conditions, and less aggressive latent tumor.

Currently the most relevant prognostic factor to predict a patient's risk of death due to PCa is the Gleason score of the biopsied tissue samples. However, pathological analysis is subjective, and the Gleason score is only a qualitative measure of the cancer malignancy.

### SUMMARY

Instead of measuring the concentration change of biomolecules as biomarkers, it is hypothesized that the detection and analysis of biomolecular interactions that occur in biological system in vivo or in vitro can be a valid approach for biomarker and therapeutic target discovery. Disclosed herein are several examples on how to utilize the interactions between biomolecules for the detection, diagnosis, prognosis, and treatment of cancer. Moreover, although the examples presented at the following are focused on cancer, one skilled in the art equipped with the teachings herein can apply the methodology other types of disease.

Also, in a specific implementation of the unique interactions involving tumor/cancer molecules disclosed herein is a simple nanoparticle test that may be used to quantitatively evaluate the PCa aggressiveness. This new test is based on a novel nanoparticle-enabled dynamic light scattering assay (NanoDLSay™) as illustrated in FIG. 1. This technique detects target analytes by monitoring the gold nanoparticle (AuNP) size change upon adsorption or binding of biomolecules to the nanoparticles using dynamic light scattering (DLS). Biomolecules such as proteins and DNAs are large molecules. Their dimension is typically in the range of at least a few nanometers. Upon specific binding or non-specific adsorption of biomolecules to the AuNPs (only non-specific adsorption is shown here as an example in the illustration), the AuNP size will increase, or the AuNPs will form clusters due to protein crosslinking. Either case will lead to an increase of the average particle size of the assay solution. Such size changes, readily detected by DLS, can reveal the biomolecule concentration, dimension or other information. This technique has been successfully applied for a wide range of applications, including protein and DNA detection, protein-protein interaction study, and protein complex detection and binding partner analysis.

### BRIEF DESCRIPTION OF THE DRAWINGS

FIG. 1. An illustration of nanoparticle-enabled dynamic light scattering assay (NanoDLSay™) for biomolecule detection and analysis. The binding of proteins to the gold nanoparticles (AuNP) through specific or non-specific interactions will lead to the formation of a protein corona on the nanoparticle surface, or nanoparticle clusters. In both cases, the average particle size of the assay solution will increase compared to the AuNP probe solution and such changes can be readily detected using DLS. In addition to proteins, other types of biomolecules such as DNAs, RNAs, or polysaccharides may also adsorb or bind with AuNPs. The interactions between the AuNPs and biomolecules can be specific antibody-antigen interactions (in this case, a specific antibody is first immobilized on AuNPs to form an immunoprobe); or non-specific adsorption interactions such as electrostatic interactions, Au—N, and Au—S bonding.

FIG. 2. The average particle size of the assay solution after 8 min of serum-AuNP incubation. 8 serums samples (healthy donor=4; BPH=4) were spiked with 4 prostate tissue lysates from normal, tissue with Grade 1, Grade 2, and Grade 3 prostate adenocarcinoma. The Gleason scores of the three tumor tissues are: 4(2+2), 5(2+3), and 9(5+4), respectively. A: the scatter-plot of all 32 samples; B: an expansion of A with 6 samples that have relatively smaller average particle sizes; C: a column-plot version of A; D and E: two sets of assay results of two different serum samples spiked with tissue lysates from normal healthy donors, BPH patients, and PCa donors. The tumor grade of the PCa donor samples are listed in each figure, respectively. In the first set, PCa2 tissue lysate was spiked to the serum with two different ratios: 1:20 and 1:100 (tissue lysate:serum, v/v). F: the assay results of one serum sample spiked with four sets of matched lung tissue lysates (N: normal, T: tumor) and the pure serum without tissue lysates.

FIG. 3. AuNP adsorption study of pure huIgG (1 mg/mL) solution spiked with normal and prostate tumor tissue lysates. PCa1 and PCa2 are the same Grade 3 tumor tissues used for the study in FIG. 2D.

FIG. 4. Average particle size of the assay solutions of healthy donor serum samples (male=12; female=12) upon adsorption to AuNPs.

FIG. 5. Illustration of huIgG-dimer induced AuNP cluster formation and antigen-bound huIgG complex adsorption to AuNPs.

FIG. 6 is graph showing nanoparticle adsorption assay results involving one embodiment showing an inverse correlation between the average particle size of the assay solution and the tumor grade.

FIG. 7 pertains to graphs showing the results of average particle size determinations of nanoparticles exposed with IgG samples (FIG. 7A) or nanoparticles exposed to IgG-tissue lysate samples (FIG. 7B).

FIG. 8 is a graph showin the measured particle sizes of five samples from prostate cancer patients, and five samples from normal and BPH patients.

FIG. 9 pertains to graphs showing the results of average particle size determinations of nanoparticles exposed with IgG samples spiked with normal or tumor tissue lysates (FIG. 9A) or nanoparticles exposed to IgG samples spiked with serum from normal or tumor possessing subjects (FIG. 7B).

FIG. 10 pertains to a graph showing the difference between IgG samples spiked with tumor vs. normal breast tissue samples.

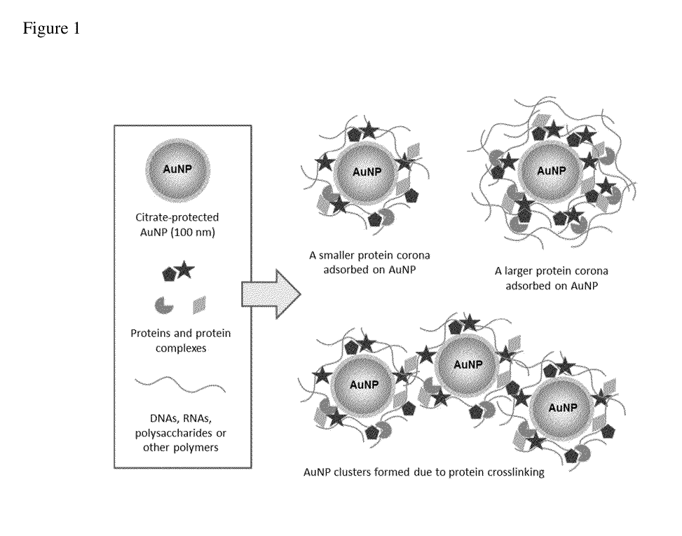

### DETAILED DESCRIPTION

According to one embodiment, provided is a method of determining a presence, and/or aggressiveness level, and/or response to therapy of a human disease in a subject. The method includes determining the size of nanoparticles upon being exposed to a biological sample or a component of biological sample or pretreated biological sample from the subject to form an assay solution, wherein the average particle size of the assay solution is correlative to the presence, and/or aggressiveness level, and/or response to therapy of disease in said subject. In a further embodiment, the human disease is cancer. In a specific embodiment, the nanoparticle used is a gold nanoparticle. In another specific embodiment, the determining step is conducted via dynamic light scattering. Alternatively, the biological sample is untreated or pretreated with a chemical or biological substance before being exposed to the said nanoparticles.

In a further embodiment, provided is a method of determining a presence, and/or aggressiveness level, and/or response to therapy of a disease in a subject. This method embodiment involves exposing a biological sample or a component of the biological sample or pretreated biological sample from the subject to a chemical or biological substance to produce a chemical interaction product, and analyzing the chemical interaction product upon such exposure. The chemical interaction product may be, but is not limited to, an enzyme-substrate interaction product. In a specific embodiment, the enzyme is a protease.
The chemical or biological substance may be, but is not limited to, an enzyme substrate or drug molecule. In a specific example, the drug molecule is Avastin.
In another specific embodiment, the enzyme substrate includes but is not limited to a peptide, carbohydrate, DNA, RNA and/or synthetic molecule. In a specific example, the chemical interaction product pertains to a biomolecular complex. Even more specifically, the biomolecular complex includes, but is not limited to, Fc gamma receptor, and/or human or nonhuman immunoglobulin G (IgG).
In a specific example the analyzing step occurs by determining the size of nanoparticles upon being exposed to the chemical interaction product. Alternatively, the analyzing may involve conducting mass spectroscopy, immunoassay, UV-Vis absorption spectroscopy, fluorescence spectroscopy or microscopy, and/or chromotagraphy.

According to yet another embodiment, a method is provided for treating cancer that involves inhibition of proteolysis of immunoglobulin G (IgG). In an alternative embodiment, a method is provided for treating cancer involving the administration of an engineered immunoglobulin G (IgG) that is not subject to proteolytic breakdown to a subject.

Also disclosed is a method for treatment of cancer that involves inhibition of matrix metalloproteases followed by or simultaneously used with anti-cancer drug treatment.

Furthermore, disclosed is a method for prevention and treatment of cancer that involves the delivery of human IgG from pooled human blood or its derivative product to a subject.

According to an additional embodiment, provided is a method to determine the effect of a drug candidate. This method involves exposing the drug candidate to a biological sample or a component of biological sample, and analyzing the interaction product of the drug and the biological sample. In a specific example, the analyzing step is conducted by exposing the mixture to a nanoparticle solution, followed by measuring the average particle size of the solution, and the size is correlative to the effect of the drug.

According to a further embodiment, disclosed is a method of determining a presence of or aggressiveness level of prostate cancer in a subject. The method may involve determining a size change of nanoparticles upon being exposed to a biological sample. The biological sample may pertain to blood and/or blood component such as plasma/serum. A change in size of the nanoparticles upon being exposed to the biological sample is directly correlative to the presence of or aggressiveness level of prostate cancer in the subject. In a typical embodiment, determining the size change involves the use of dynamic light scattering. A decrease in nanoparticle size indicates prostate cancer in the subject.

In a more specific embodiment, the greater the decrease in nanoparticle size correlates with a higher aggressiveness level of the prostate cancer. Standards for size changes based on normal tissue samples as well as samples from cancer tissue samples with known aggressiveness are developed using the techniques taught herein. These standards can be used to grade the size differential of a test sample, and can be helpful to correlate the degree of aggressiveness of the test sample.

The term “biological sample” as used herein refers to a sample obtained from a biological source, including human or nonhuman animals, plants, or microorganisms. Biological samples may include but are not limited to, a biological fluid, tissue sample, cell sample, tissue lysate, or components thereof obtained from an animal. Biological fluids may include but are not limited to blood or blood components (e.g. serum), saliva, tears, sweat, vaginal discharge, mucous, semen, urine, gastric fluid, bile, or feces including. A biological sample may also include blood or blood component spiked with a tissue lysate from the subject. Typically, but not necessarily, the blood or blood component sample is incubated with the tissue lysate for a period of time to allow biomolecules to interact.

As used herein, the term “subject” refers to a human or nonhuman mammal. In a typical embodiment, the subject is a human suspected of having disease or health condition.

In another typical embodiment, the nanoparticles used pertain to, but are not limited to, a metal nanoparticle such as gold and/or silver containing nanoparticles. Alternatively, nanoparticles made of other materials may be used, particularly if the nanoparticles are able to scatter light. In a more specific embodiment, the gold or silver nanoparticles possess an average size with a size deviation of 50 nm from the average. In an even more specific embodiment the average size of the nanoparticles is 10-1000 nm. In a further specific embodiment, the average size of the nanoparticles is 50-500 nm.

According to another embodiment, the invention is directed to a method of identifying a prostate cancer biomarker. The method involves determining a size change in nanoparticles upon being exposed to a biological sample. The biological sample includes blood or a blood component spiked with a biomolecule sample from prostate tumor tissue. Biomolecule is defined as a chemical or ionic species that exists in a biological system. Typically, the biomolecule is a protein. The effect of a significant size differential indicates that the biomolecule in the biomolecule sample is a potential biomarker.

Furthermore, another embodiment involves the identification of blood molecules specifically interacting with the prostate cancer tissue sample, may in turn be used in diagnostic techniques or research involving the mechanisms involved in the cancer process. In a specific example, the blood biomolecules are human IgG (huIgG).

According to an additional embodiment, the disclosure is directed to a treatment of prostate cancer. The method involves the administration of a therapeutically effective amount of blood biomolecules from the patient or from an allogeneic source.

According to a further embodiment, the disclosure pertains to a method of determining an aggressiveness level of prostate cancer in a subject. The method involves determining a size change of nanoparticles upon being exposed to a biological sample that includes blood or blood component from the subject spiked with a prostate tumor tissue sample. The greater the decrease in nanoparticle size the higher the aggressiveness level of the prostate cancer is. This can be determined qualitatively or quantitatively. Also, standards can be utilized to compare the size differential with that of tissue samples of a known condition, whether normal or at a certain tumor stage.

In another specific embodiment, disclosed is a new assay method for biomarker detection. The method involves use of a specific or non-specific probe or material to catch huIgG molecules, and then use of a specific antibody or other binding molecules to analyze the target biomarker molecules bound to huIgG. The probe for catching human IgG and the specific target biomarker may be used simultaneously to detect the biomarkers bound to human IgG. The same assay format can be applied to other serum protein-tumor biomarker complexes that exist in the blood.

### EXAMPLES

### Example 1

### Assay for Detecting Effect of Tumor-Associated Chemicals and Biomolecules on Nanoparticle Interactions

In a most recent study, using NanoDLSay™, it was discovered that multiple molecular aberrations from mouse and human blood serum samples with and without prostate cancer. In the analysis, a serum sample was simply mixed with a citrate-protected AuNP solution to allow proteins and possibly other biomolecules to adsorb to the AuNPs. Of the most relevance to the work reported here, it was found from the previous study that there is a significant difference in the serum-adsorbed AuNP size between mouse serum samples with and without prostate tumor. The average particle size of the assay solution is substantially smaller for mice carrying large tumor grown from orthotopically injected PC3 cells compared to healthy control mice and mice bearing smaller tumor grown from LnCaP cells. However, from the previous study, a significant difference from human serum samples with and without prostate cancer was difficult to observe.

The biggest challenge for cancer biomarker research and early cancer detection is that at early stage, the amount of specific molecules that are released from the tumor to the peripheral circulation system is very small. In the mice model study conducted previously, three groups of mice were prepared: mice with large tumor grown from aggressive PC3 cells, mice with smaller tumor grown from less aggressive LnCaP cells, and normal healthy controls. The relative tumor mass versus body weight of the PC3 and LnCaP mice was approximately 5% and 0.3%, respectively. These ratios would correspond to a tumor mass of 2.5 Kg and 150 g in a human patient with a body weight of 50 Kg. Such tumor size is far exceeding the tumor size from human patients with early stage cancer. It is not surprising that the difference found from mice models was not observed from human serum samples.

One aspect disclosed herein is a spiking experiment for human serum study. A prostate tissue lysate sample is spiked into a human serum sample and then the serum sample is subjected to AuNP adsorption test. It is hypothesized that when tumor develops in the human body, some unique chemicals or biomolecules are released from the tumor to the blood, causing certain serum molecular changes to occur and such molecular changes are reflected in the AuNP adsorption assay. By spiking a tumor tissue lysate directly to the blood, the concentration of tumor-associated chemicals or biomolecules in the blood serum is synthetically increased, and as a result, molecular change of the blood serum similar to what occurs in vivo may be observed.

It has now been discovered that the difference from human serum samples with and without prostate cancer can be observed: the average particle size of human serum samples spiked with prostate tumor tissue is significantly smaller than the serum samples with normal tissue lysates. More importantly, there is a quantitative, inverse correlation between the nanoparticle size and the grade of the prostate tumor. The molecular mechanism behind the observed nanoparticle size difference between tumor and normal tissue-spiked serum samples is also elucidated. There appears to be an immune reaction between molecules released from the prostate tumor tissue and human IgG. This interaction changes the adsorption of human IgG to the AuNPs, leading to the observed nanoparticle size difference. More interestingly, the molecular mechanism proposed herein provides possible explanations to several long-standing questions in the general area of cancer: why prostate tumor is a slow-growing tumor while lung cancer is more aggressive; why younger males tend to have more aggressive prostate cancer than older males; and why females tend to have significantly lower cancer rate than males in most cancer categories. The molecular mechanism further suggests that a prostate cancer patient's own IgG may be the best potential drug for his treatment.

Results

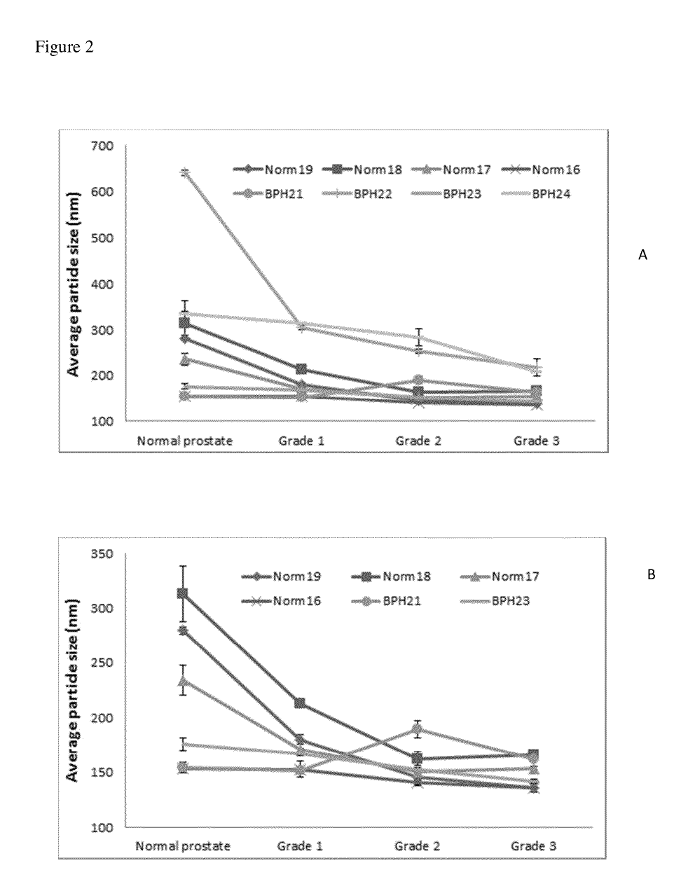

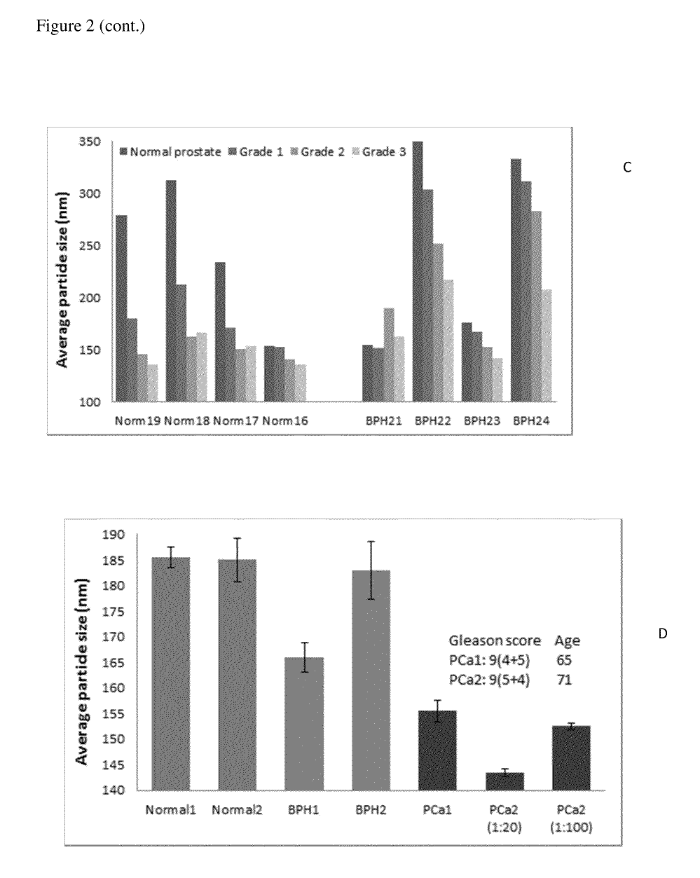

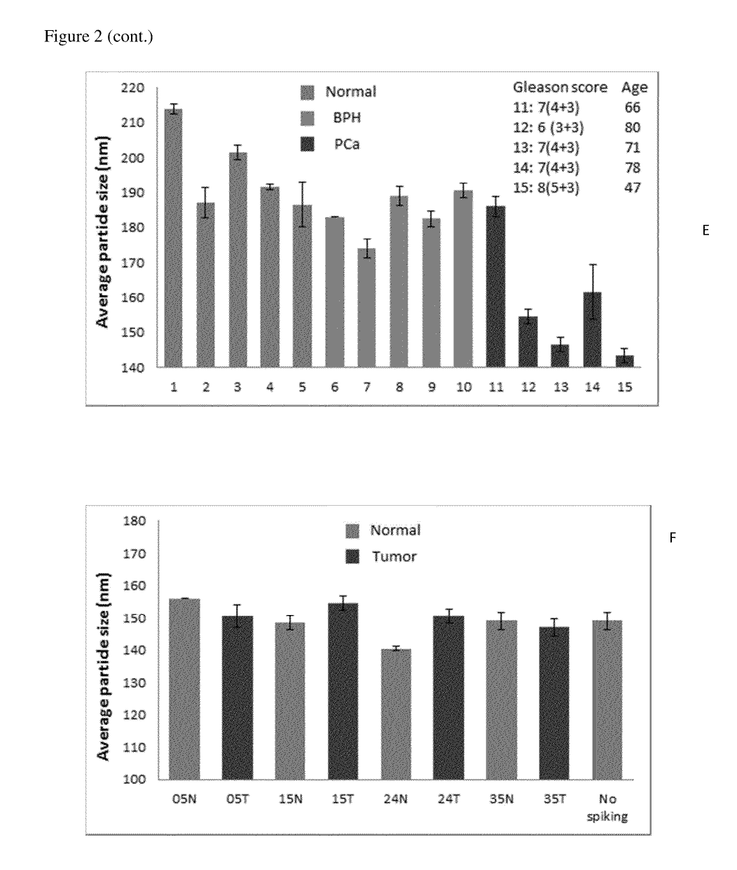

In a first set of experiments, 8 male serum samples spiked with 4 different tissue lysates (total 32 samples were prepared) were tested. Among the 8 serum samples, 4 were from normal healthy donors and 4 from patients with BPH (benign prostate hyperplasia). The 4 prostate tissue lysates are from normal healthy control, tissue with Grade 1, Grade 2, and Grade 3 prostate adenocarcinoma. All tissue lysates were prepared in exactly the same buffer using the exactly same protocol. The final total protein concentration of all tissue lysates was adjusted to 1 mg/mL. FIG. 2A-C is the measured average particle size of the assay solution at 8 min of serum-AuNP incubation: A is the scatter-plot of all 32 samples; B is an expansion of the 6 samples with relatively smaller average particle sizes; and C is a column-plot version of A. Among the 8 sets of serum samples, 7 sets exhibited a clear trend of decreased average particle size when the serum was spiked with prostate tumor tissue lysates. The average particle size is inversely related to the grade of the tumor tissue. In addition to the data presented here, additional sets of normal and tumor tissue lysates-spiked serum samples (including data presented here, total approximately 80 samples made from the combination of 10 serum samples spiked with 8 different tissue lysates) at different time were tested, and all showed the same trend. BPH21 serum is the only exception observed throughout the whole study.

In a second set of experiment, the different particle size of a serum sample spiked with normal, BPH, and PCa tissue lysates, respectively, were compared. Two sets of serum samples were prepared and analyzed, with data presented in FIGS. 2D and E, respectively. In the first set of samples, the two PCa tissue lysates are both from Grade 3 tumor: PCa1 has a Gleason Score of (4+5) and PCa2 has a Gleason Score of (5+4). Again, the spiking of PCa tissue lysates to the serum led to a much smaller average particle size of the assay solution. As to the two BPH tissue lysates, one behaved like the normal tissue, and another one caused the particle size decrease of the assay solution, however, the decrease is smaller than the PCa tissue lysates. There is also a difference between the two PCa tissue samples: PCa2 caused more substantial particle size decrease than PCa1, even though PCa1 has a Gleason score of (5+4) while PCa2 has a Gleason score of (4+5). These two tissue lysate samples were also spiked into other serum samples, and the same difference between the two was observed from all serum samples tested. In contrast to the pathological analysis, the AuNP adsorption assay conducted here suggests that PCa2 is more aggressive than PCa1. From this experiment, a concentration-dependent effect was observed: PCa2 tissue lysates were spiked into the same serum in 1:20 and 1:100 (lysate:serum, v/v) ratio, respectively. With an increased amount of tissue lysate spiked into the serum, the particle size decreasing effect caused by the tumor tissue lysate is more significant. The second set of serum samples presented very similar results (FIG. 2E): in general, the BPH tissue lysate-spiked samples showed very slight nanoparticle size reduction, while 4 among the 5 Grade 2 PCa tumor tissue lysate-spiked samples showed substantial nanoparticle size reduction compared to the normal tissue lysates. Also worth of special attention, the more aggressive tumor among the five samples, #15 from a donor of age 47 with a Gleason score of 8, showed the largest nanoparticle size reduction.

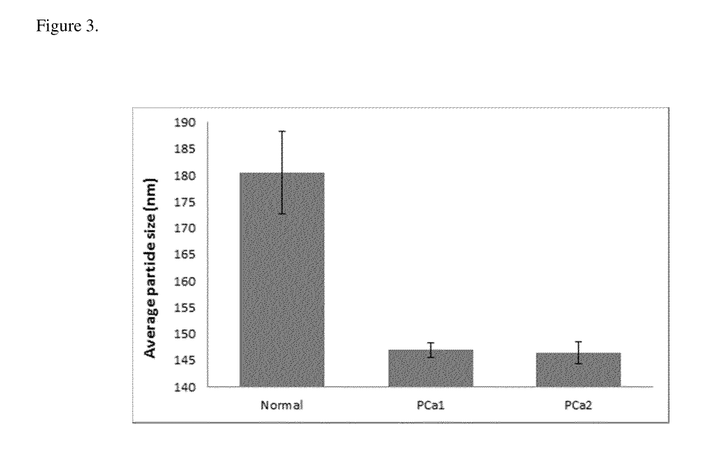

In a third set of experiment, the same analysis on matched normal and tumor lung tissue lysates-spiked serum samples was conducted as a comparison to the prostate tissue study. The purpose is to see if the observed nanoparticle size reduction from tumor-spiked serum samples is unique to prostate cancer or is a general phenomenon in other types of cancer. All the lung tissue lysates were prepared using the exact same protocol as prostate tissue lysates, and the final total protein concentration of all lung tissue lysates was 1 mg/mL. Because of the relatively large size of lung, matched lung tissue samples can be obtained and used in the study: the matched tumor sample and the “normal” sample were obtained from the same donor that has been diagnosed with lung cancer. However, the “normal” tissue was taken from a location of the lung as far as possible from where the tumor is located, and was confirmed to be normal cells by pathology analysis. Four sets of matched lung cancer tissue lysate samples were tested, each set diagnosed with different lung cancer: adenocarcinoma (05N and 05T), large cell (15N and 15T), small cell (24N and 24T), and squamous cell lung cancer (35N and 35T). Total three sets of serum samples were used in this study and the result of one set of data is presented in FIG. 2F. No significant difference was found between the matched tumor and normal samples, and pure serum (no tissue lysate spiking) from all three sets of serum samples. To understand the possible interactions between the tissue and serum proteins that have caused the observed differences between prostate tumor and normal tissues, the same assay on human IgG (huIgG) solutions spiked with normal and tumor tissue lysates was conducted. Human blood consists more than thousands of proteins. It is believed that the proteins adsorbed to the AuNPs are primarily serum-abundant proteins such as huIgG. The typical concentration of huIgG in blood serum is around 5-15 mg/mL. Immunoglobulin (IgG) proteins are known to have strong affinity towards AuNPs, and as a matter of fact, this property has been used commonly by diagnostic industry to prepare AuNP immunoprobes through a simple adsorption process. It was frequently observed in the noted study that even at high ng/mL to low μg/mL concentration range, IgG, including huIgG, can be completely adsorbed to AuNP within a few minutes. Three huIgG solution at a concentration of 1 mg/mL spiked with a normal, and two grade 3 prostate tumor tissue, PCa1 and PCa2 (same as used in FIG. 2D experiment) were prepared. The assay was conducted under exactly the same condition as applied to the serum studies presented in FIG. 2. Remarkably, observed was a very interesting, similar difference between normal and tumor tissue lysate-spiked samples (FIG. 3): the average particle size is substantially smaller for tumor tissue lysate-spiked than the normal lysate-spiked solution huIgG solution. This result revealed a very interesting possibility that there may be an immune response between prostate tumor-associated proteins and biomolecules in the blood.

Discussion

Many proteins are known to adsorb to AuNPs readily through electrostatic interactions, Au—N and Au—S bonding with high affinity. It is commonly known that when citrate-protected AuNPs are mixed with blood serum, a protein layer will be adsorbed to the AuNPs and stabilizes the AuNP in the high salt content blood serum. A recent study by Doborovolskaia et al identified more than 60 proteins from blood plasma adsorbed to citrate-protected AuNPs. Human blood consists of more than thousands of proteins. Initial thoughts of trying the AuNP-blood serum adsorption assay for cancer biomarker discovery was based on a simple hypothesis that there may be some differences in the proteins adsorbed to AuNPs between cancer and non-cancer serum samples. Indeed, it was observed that a substantial difference existed in the average particle size of serum-adsorbed AuNPs between tumor-bearing mice and healthy control mice from a previous study. Now by introducing a spiking experiment to synthetically increase the concentration of tumor-associated chemicals and biomolecules in human blood serum, the same phenomenon as found from mice models was observed. More importantly, a quantitative, reverse correlation between the nanoparticle size of the assay solution and prostate tumor grade was found. All tissue lysate samples, cancer and normal samples used in this study were prepared under the exact same conditions using the same buffer solution (a modified RIPA buffer).

The observed difference between normal and prostate tumor tissue lysates is not due to the buffer effect. Serum samples spiked with the modified RIPA buffer used for tissue lysate preparation were tested and no difference was observed from the spiked and un-spiked samples. The difference is also not due to the different total protein concentration among different samples, since all lysates have the same total protein concentration. Data presented in each figure were obtained from the sets of serum and tissue lysate samples that were stored, thawed, and assayed under exactly the same conditions at the same time. The reproducibility of the assay is excellent, as evidenced from the general low standard deviation of each assay (CV % less than 10% for all data). Furthermore, the tumor tissue-induced nanoparticle size reduction was observed from prostate tissue lysate samples, but not from lung tissue lysate samples.

Based on various control and comparison studies, it is believed that the reduced nanoparticle size observed from tumor tissue lysate-spiked serum sample is caused by the chemicals or biomolecules associated with prostate tumor tissue. A key question is then how the chemicals and biomolecules released from the prostate tissue changed the proteins adsorbed to the AuNPs in blood serum.

Not to bound by any particular theory, there are two most likely possibilities. One is that certain molecules from the tumor tissue interact with the abundant serum proteins, possibly even trigger some biochemical reactions, and subsequently change the binding activity of the serum proteins to AuNPs. A second possibility is that the unique molecules released from the tumor tissue directly compete with other serum proteins to bind with the AuNPs. The tumor-associated proteins and molecules are for some reasons smaller than those from normal tissue, and also smaller than serum proteins. The competitive binding of these “smaller” proteins and molecules caused the observed nanoparticle size reduction. Among the two possibilities, it is believed that the first one is a more likely scenario than the second one.

In the preparation of tissue lysate-spiked serum samples, 1 μL tissue lysate at a total protein concentration of 1 mg/mL was added into 20 μL of serum sample. The typical total protein concentration of a blood serum is around 50-100 mg/mL. This means the total protein amount added from tissue lysates to the serum is less than 1/1000 of the total protein concentration of the serum, and the concentration of each individual protein will be even lower. It is unlikely that the low concentration proteins from the tumor tissue lysate will be able to compete with the abundant serum proteins to bind with AuNPs, and cause particle size reduction as dramatic as seen from this study. Furthermore, a protein-AuNP adsorption study with pure tissue lysates was conducted. The assay revealed that the average particle size of the tissue protein-adsorbed AuNP is actually slightly larger for most tumor tissue lysates than for normal tissue (one example is: average particle size of 145 nm for a Grade 3 tumor tissue and 136 nm for a normal tissue lysate). The next question is then what protein or proteins from the serum have been changed by the added tissue samples, and what is the nature of their interactions. Among the abundant serum proteins, it was conjectured that this was human IgG. This consideration was based on the high binding affinity of huIgG to AuNPs, as mentioned earlier, and also the high concentration of huIgG in blood (average 5-15 mg/mL).

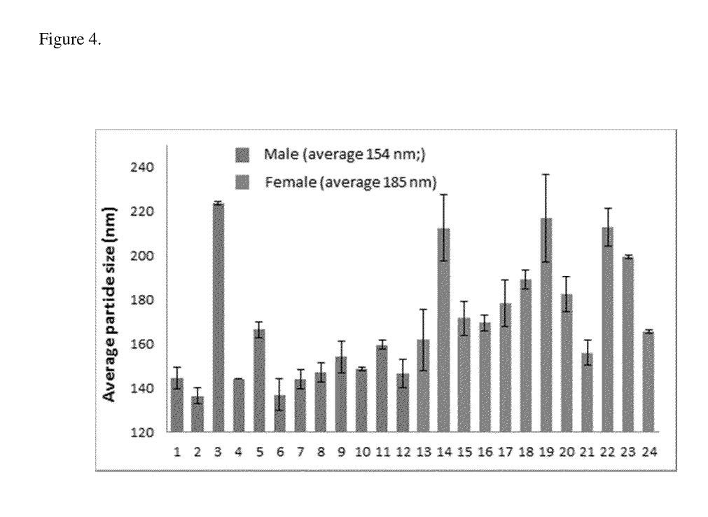

Remarkably, from the tissue lysate-spiked pure huIgG protein solution study as shown in FIG. 3, the exactly same nanoparticle size reduction from tumor tissue-spiked solution was observed. All the evidence so far suggests that there is an immune reaction between huIgG and prostate tumor. In other words, there is an immune defense in the blood against the prostate tumor spread. This suggestion was further supported by another two side evidences. Lung cancer is known to be a much more aggressive tumor than prostate cancer. The comparison study conducted on lung tumor tissue samples suggests that such an immuno-defense reaction is lacking in the blood for lung cancer. Once a lung tumor is formed, the chemicals or tumor cells may be freely transported to other sites of the body through the blood, enabling tumor metastasis. A second side evidence is that women are known to have lower cancer rate than men in almost all types of cancer, except female reproduction organ-related cancer such as ovarian cancer and breast cancer. Age-matched 12 serum samples from healthy male donors and 12 serum samples for healthy female donors in the AuNP test was tested. It was found that the average nanoparticle size of male samples is significantly smaller than female samples (FIG. 4). Females are known to produce more antibodies, since they produce anti-male antibodies. It is also known that females who have given birth to children have a less tendency to have cancer, because more antibodies are produced during child-birth.

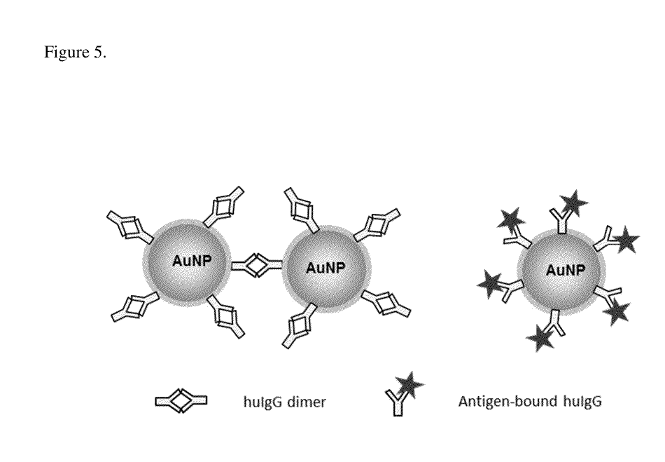

Combining all of the relevant evidence, a mechanistic model as shown in FIG. 5 is proposed to explain the nanoparticle size reduction observed from prostate tumor tissue-spiked serum samples. huIgG exists in a dimer form in pure solution or in a non-antigen binding state. This claim is supported by direct size measurement of huIgG using dynamic light scattering. The measured size is 13-14 nm for huIgG at concentration of 1 and 10 mg/mL. This size indicates that huIgG is indeed dimerized. This claim is also supported by results that were published previously on the interaction study between a protein A-conjugated AuNP and huIgG. From that study, it was found that at saturated binding level, the net size increase of the AuNP is approximately 40 nm. This size does not correspond to IgG monomer, instead, corresponds to a huIgG dimer. At the presence of huIgG dimer, the AuNPs can be crosslinked together into clusters, which subsequently increase the nanoparticle solution substantially. Upon binding with antigen, for example, prostate tumor tissue-associated antigen, the huIgG dimer is broken, and the antigen-huIgG complex lacks the capability to cross link AuNPs. However, they can still bind to the AuNPs. As a result, the average particle size of the assay solution is reduced.

In summary, it is demonstrated herein that a simple nanoparticle test of blood serum spiked with tissue-derived samples can provide quantitative information on PCa tumor grade and aggressiveness. The spiking experiment synthetically increased the concentration of tumor-associated proteins and biomolecules to the blood serum, making the molecular change in serum more easily detectable. More importantly, this spiking experiment allows one to detect molecular changes of blood caused by cancer without actually knowing which specific protein or proteins from the tumor have caused such changes. Assuming huIgG is indeed the serum protein that interacts with tumor tissue proteins, results presented here suggest that the human body has a natural defense system against prostate cancer, or possibly other types of slow-growing cancer. The human immune system recognizes cancer cells and related chemicals and molecules as non-self. It may be possible to purify the huIgG obtained from cancer patients and use it as an auto-produced anti-cancer drug to treat the cancer.

Methods and Materials

Materials:

Gold nanoparticles (AuNPs) used in this study (15708-9) was purchased from Ted Pella Inc. (Redding, Calif.). The average diameter of the AuNP solution is 100 nm and the concentration of the nanoparticle is 10 pM. The four pure proteins used in the study, huIgG (ab91102), α2M (ab91104), PSA (ab78528) and PAP (ab96164), were all purchased from Abcam (www.abcam.com). All human serum samples were purchased from Asterand Solutions (www.asterand.com). Tissue lysate samples were purchased from Protein Biotechnologies (www.proteinbiotechnologies.com). All serum and tissue lysate samples were stored at −80° C. for long-term storage before shipping. Upon arrival, the samples were thawed, aliquoted and stored at −20° C. before assay was conducted. All human tissue and serum samples used in this study were de-identified, archived specimens. University of Central Florida IRB committee approved the use of these commercially acquired samples with IRB exemption. For tissue lysate preparation, tissue specimens are homogenized in a modified RIPA buffer to obtain the soluble proteins, and centrifuged to clarify. All lysate solutions were adjusted to have a total protein concentration of 1 mg/mL using the same buffer. The composition of the modified RIPA buffer is the following: PBS (pH 7.4), 1 mM EDTA, 0.25% Na deoxycholate, 1 mM Na₃VO₄, 1 mM NaF, 0.1% SDS, 1 mM PMSF, 1 μg/mL aprotinin, 1 μg/mL pepstatin-A, and 1 μg/mL leupeptin. The lysates have not been subjected to denaturing or reducing conditions. Dynamic Light

Scattering Analysis:

Particle size analysis of the assay solutions was conducted using an automatic DLS instrument, NDS1200, from Nano Discovery Inc. (Orlando, Fla., www.nanodiscoveryinc.com). This system is equipped with a 12-sample holder carousel to allow automatic measurement of 12 samples. The measurement error for the pure AuNP solution with an average diameter of 100 nm is ±2 nm. The sample cell is a disposable, 4.8 by 30 mm cylindrical glass tube. The measurement time for each sample was set as 20 s and additional 10 s of delay was set to allow a total 30 s of delay before next sample was measured.

Sample Preparation and Assay Methods:

All nanoparticle assays were conducted by adding 2 μL of sample solution to 40 μL AuNP solution. The sample incubation time varies slightly from one experiment to another, and is specified in each figure separately. All assays were conducted in replicates and data presented in each figure is the average of the replicates. The error bars are standard deviations. To prepare tissue lysate-spiked serum samples or pure protein solutions, 1 μL lysate solution was mixed with 20 μL serum or pure protein solution. The mixed solution was set at 4° C. overnight before nanoparticle assay was conducted. To conduct the tissue lysate-AuNP adsorption study, 2 μL tissue lysate was directly mixed with 40 μL AuNP solution, and assayed under the same condition as used for the serum-AuNP adsorption analysis.

### Example 2

### Tumor Tissue-IgG Interaction Reveals Prostate Cancer Status and Aggressiveness

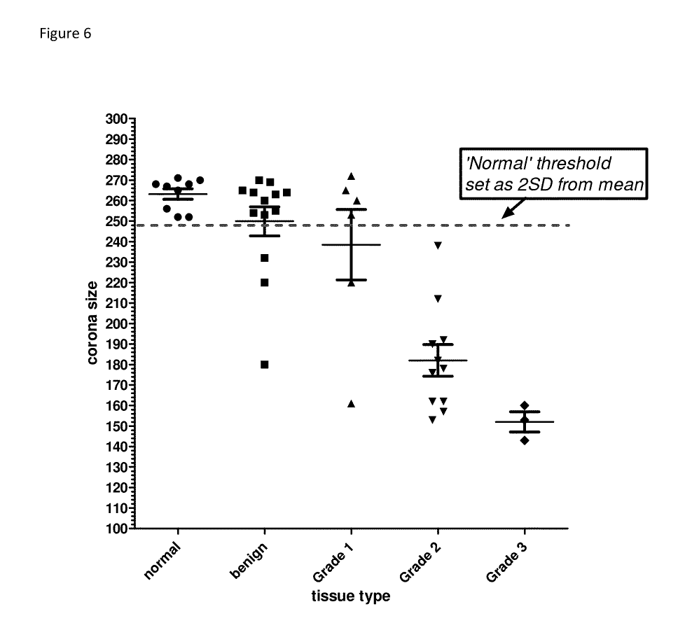

It has been discovered that prostate tumor tissue contains certain molecules that can interact with human immunoglobulin G (IgG) in the blood. The gold nanoparticle (AuNP) adsorption assay was conducted on pure human IgG solution spiked with prostate tissue lysates. Human IgG (cat. ab91102) from Abcam (www.abcam.com) was used for this study. Total 42 prostate tissue lysates, including normal (n=9), BPH (n=13) and prostate cancer samples (n=20) with different tumor grades, were spiked into a pure human IgG solution (1 μL lysate at a total protein concentration of 1 mg/mL to 20 μL of IgG solution at 1 mg/mL). After incubation for 30 min, and the spiked samples were subjected to the gold nanoparticle adsorption assay. To conduct the assay, 2 μL of spiked sample was mixed with 40 μL of AuNP solution (d, 100 nm). FIG. 6 is the nanoparticle adsorption assay results: there is an inverse correlation between the average particle size of the assay solution and the tumor grade. The most aggressive Grade 3 tumor can be clearly differentiated from normal tissue without any overlap. Most benign and Grade 1 tumor tissues gave similar results as normal tissues, but with two samples resembling a more aggressive tumor profile. The assay results of 11 Grade 2 tumor tissues extend over a wide range, reflecting exactly the ambiguous aggressiveness of the Grade 2 tumor. According to the pathological reports, all these Grade 2 tumors are the same or similar; however, based on the nanoparticle test, there are substantial differences between these intermediate grade tumors, suggesting that they should have been treated differently. This study revealed that there is a molecular interaction between prostate tumor tissue and human IgG and such interactions are indicative of prostate tumor status and aggressiveness. Such interactions also changed the adsorption behavior of IgG with gold nanoparticles.

### Example 3

### Fc Gamma Receptor Binding with Human IgG is Involved in Prostate Cancer

Human IgG can have molecular interactions with many biomolecules. To explain the results observed in Example 2, it was first hypothesized that Fc gamma receptor is one molecule from the tumor tissue that contributed to the tumor tissue-IgG interactions. The overall function of Fc gamma receptors is to bind with the Fc region of IgG and immunocomplexes as part of the immune functions. When the Fc region of the IgG dimer is blocked by the Fc gamma receptors, the IgG dimer can no longer crosslink the AuNPs. There are five major Fc gamma receptors (FcγRs) with closely related functions. To confirm the above hypothesis, several experiments using two Fc gamma receptors, FcγRI (CD64) and FcγRIIB (CD32) was conducted. FcγRI is known to bind to the Fc region of IgG and its immune complexes with high affinity (Kd˜10⁻⁹M), while FcγRIIB mainly binds with aggregated IgGs with lower affinity (Kd˜10⁻⁷M).

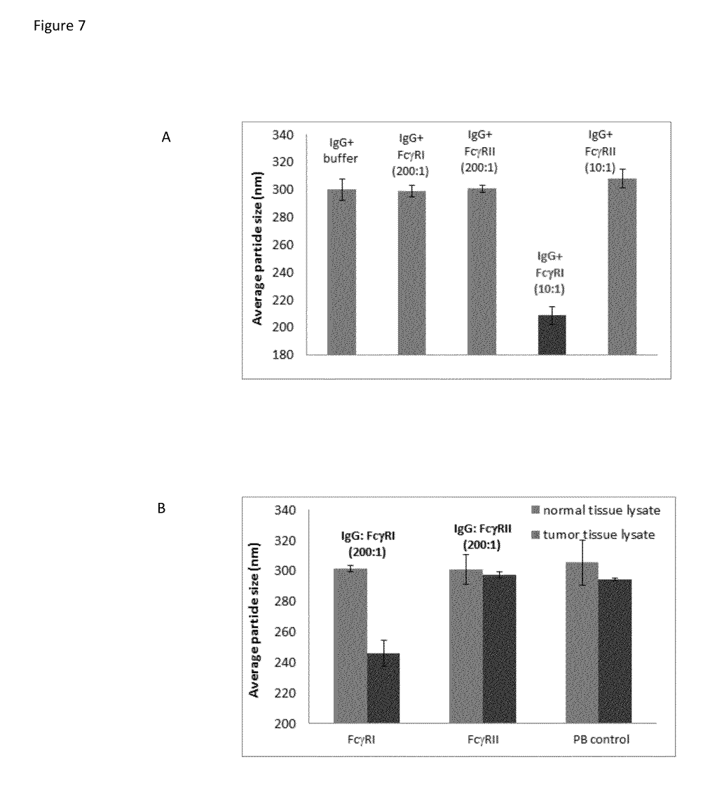

In a first experiment, the two FcγRs or a control buffer are spiked into IgG solution (ratios shown in FIG. 7A are IgG:FcγR protein weight ratio). The spiked IgG solutions were then subjected to AuNP adsorption analysis. Among all the samples, IgG spiked with FcγRI at 10:1 protein ratio showed significant particle size reduction, while IgG spiked with the same amount of FcγRIIB did not show particle size reduction (FIG. 7A). It should be noted here that FcγRI (44-55 KDa, SDS-Page) is a slightly larger protein than FcγRIIB (25-35 KDa). The direct adsorption of FcγRI and FcγRIIB to the AuNPs was conducted; FcγRI shows a slightly larger nanoparticle size increase (increase by about 12-15 nm) than FcγRIIB (increase by about 10 nm). So the observed size reduction by the addition of FcγRI to IgG solution cannot be due to the different size of FcγRI and FcγRIIB. This first set of experiment confirmed that the interaction between FcγRI and IgG and IgG dimer can indeed lead to the reduced nanoparticle size in the IgG-AuNP adsorption assay as was hypothesized.

In a second experiment, FcγRI and FcγRIIB (20 μL at 0.1 mg/mL) or equivalent volume of phosphate buffer (PB) solution was first mixed with 2 μL of normal or tumor tissue lysate. The mixed solution was then spiked into IgG solution (1 μL mixed solution to 20 μL IgG solution at 1 mg/ml, notice here that the IgG:FcγR ratio is only 200:1), and then IgG-AuNP adsorption assay was conducted. Among the six samples (two different tissue lysates were studied for each type of sample), only the combination of FcγRI with tumor tissue lysates led to significant particle size reduction (FIG. 7B). This data, in comparison with the data shown in FIG. 7A, suggests that the tumor tissue contains certain chemicals/molecules that can activate FcγRI receptor. IgG dimer is a special form of IgG immunocomplex, idiotype-anti-idiotype complex. Since Fc receptor's function is to bind with immunocomplexes, it is reasonable to say that the FcγRI may have prevented the IgG dimer from crosslinking the AuNPs by binding with the Fc region of the IgG and IgG dimer.

These experiments confirmed that there are indeed interactions between FcγRs and IgG, and such interactions are affected by the presence of tumor. Therefore, the FcγRs-IgG interaction may be used as a biomarker for cancer detection.

### Example 3

### Over-Expressed Proteases in Prostate Cancer Serum Cause Proteolytic Cleavage of IgG

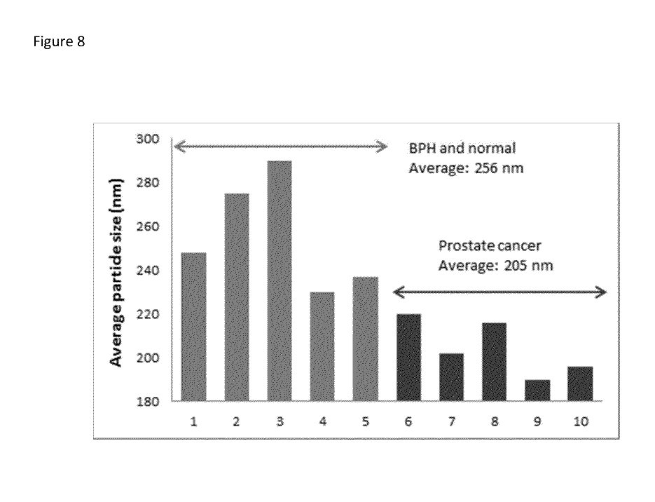

The over-expressed proteases in prostate tumor may also lead to the assay results observed in Example 2. Kallikreins are a family of serine proteases that are over-expressed in many hormone-dependent cancer including prostate, breast and ovarian cancer. Serine proteases are known to cause proteolytic cleavage of IgG. Elevated kallikrein levels were reported in the blood serum of prostate cancer patients. To examine if prostate cancer serum can cause the same effect on IgG as the prostate tumor tissue lysate does, a similar experiment was conducted. First, 2 μL serum sample was diluted into 18 μL modified RIPA buffer (from Protein Biotechnologies). The dilution using RIPA buffer is to try to lyse all the cells still present in the serum, possibly prostate tumor cells, to release potential tumor-related molecules to the solution. Then solution was incubated at room temperature for at least 30 min. Then 2 μL RIPA-diluted serum was added to 20 μL human IgG (1 mg/mL) solution. After incubating for at least 30 min, the sample was subjected to gold nanoparticle adsorption study. For the assay, 2 μL sample was mixed with 40 μL gold nanoparticle solution. FIG. 8 is the measured particle sizes of five samples from prostate cancer patients, and five samples from normal and BPH patients. Again, a very similar pattern was observed similar to observed from tissue lysate studies: cancer patient serum exhibited a smaller average particle size than the normal and BPH patients.

### Example 4

### Relationship of Protease-IgG Interaction with Lung Cancer

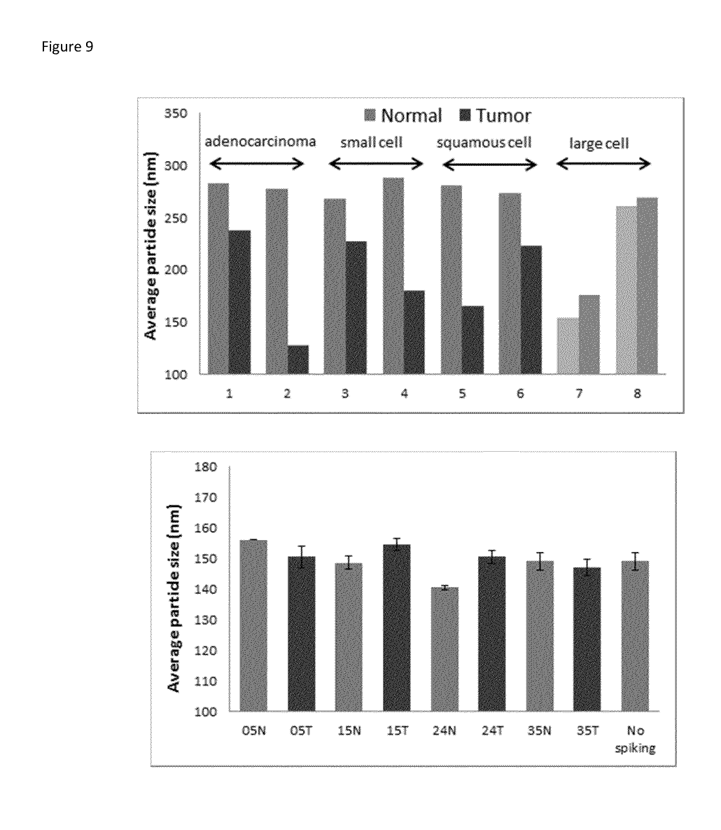

The same assay as disclosed in Example 2 was conducted on lung cancer tissue. Matched lung cancer tissues were spiked into human IgG solution under the same conditions. The results are summarized in FIG. 9A. Four types of lung cancer tissues were studied: adenocarcinoma, squamous cell carcinoma, small cell carcinoma and large cell carcinoma. Among total 8 sets of samples studied (each set contains a matched normal and lung cancer tissue), three types of lung cancers, namely, adenocarcinoma, squamous cell carcinoma, and small cell carcinoma all showed smaller size with tumor tissue-spiked samples compared to normal tissue-spiked samples, while large cell carcinoma showed opposite results: the tumor tissue-spiked sample led to larger nanoparticle size than the normal tissue-spiked sample.

When the tissue samples were spiked to human serum samples, however, no difference between normal and cancer samples was observed, which is quite different from what was observed from prostate cancer studies. The results of tissue lysate-spiked human serum samples are shown in FIG. 9B.

The difference between lung cancer and prostate cancer may be explained by the following fact: in prostate cancer, it is kallikrein family proteases that have caused the proteolytic degradation of human IgG; while in lung cancer, it is matric metalloproteinases (MMPs) that have caused the proteolytic degradation of human IgG. In blood, MMPs are inhibited by endogenous proteins such as α-macroglobulin (a serum abundant protein). Some kallikreins can be inhibited by blood serum proteins as well such as antichymotrypsin. But it is possible that not all kallikreins are inhibited by antichymotrypsin, and these kallikreins (or other proteases) caused the degradation of circulating IgG in human blood.

### Example 5

### Relationship of Protease-IgG Interaction with Breast Cancer

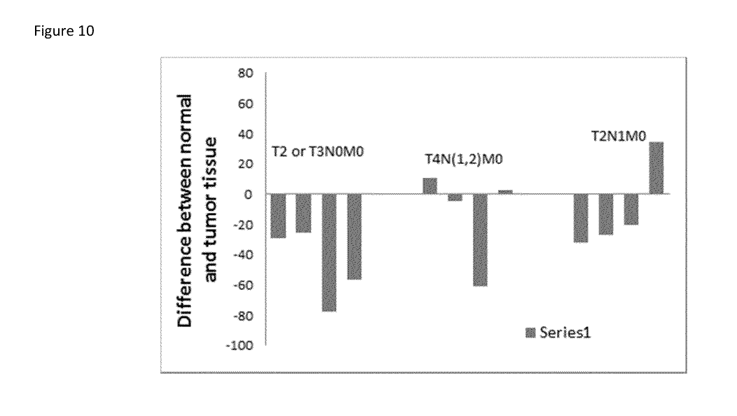

The same assay as disclosed in Example 2 was also conducted on breast cancer tissue. Matched lung cancer tissues were spiked into human IgG solution under the same conditions. The results are summarized in FIG. 10. From the results, it is demonstrated that a majority of breast tumor tissue samples (9 out of 12) show smaller particle size compared to their matched normal tissue samples; while a smaller percentage of samples (3 out of 12) show opposite effect or no difference between tumor and normal tissue samples. Data presented in FIG. 10 is the difference between the matched normal and tissue sample.

### Example 6

### Targeting the IgG Degradation for Cancer Treatment

The protease or potentially other protein-caused IgG degradation could also be used to explain why certain anti-cancer drugs such as Avastin work on lung cancer and colon cancer, but not prostate cancer. The antibody-based anti-cancer drugs are most likely subjected to proteolytic degradation by the proteases released from prostate tumor to the blood stream. The drug is destroyed before it can reach the tumor site. Avastin also appears to be not working with ovarian cancer and breast cancer. It could be that these cancers, similar to prostate cancer, release proteases that destroy the antibody drugs. In order to make the anti-cancer drug to be effective, several modifications need to be made: (1) engineer a new antibody that is not subject to proteolytic degradation; (2) protect the antibody drug from proteolytic degradation; (3) first inhibit the proteases, and proceed with the antibody drug treatment. IgG is an important part of the immune system. Many studies have found that at the early stage of cancer development, the body can use its immune system to defend cancer progression. As revealed in a related study, a tumor can continuously release proteins such as proteases and other chemicals to destroy IgG. This suggests it is necessary to restore the immune function of the system in order to slow down cancer progression. By injecting IgG from pooled human serum to a subject, this could immediately boost the immune function of the subject and to prevent or slow down cancer progression.

The proteolytic degradation of IgG may also be used to predict the drug efficacy and a patient's response to a drug. Using Avastin as an example, it can be tested whether it will work on a cancer patient by exposing a cancer patient's blood or tissue sample to Avastin, and then analyzing the potential proteolytic degradation product of Avastin using nanoparticle adsorption assay or other assay techniques. If Avastin can remain intact, that indicates the drug may be effective for this particular patient, and if Avastin is degraded, it is then not suitable for the patient. For example, it is known that some breast cancer patients do benefit from current treatment of Avastin, while most majorities do not. It is possible that the breast cancer patients that respond positively to Avastin do not have over expressed proteases in the blood that can degrade Avastin, while majority of breast cancer patients do have over expressed proteases in the blood that degrade Avastin. By conducting the degradation test of Avastin, this enables identifying cancer patients that will most likely benefit from Avastin treatment.

It should be borne in mind that all patents, patent applications, patent publications, technical publications, scientific publications, and other references referenced herein are hereby incorporated by reference in their entirety to the extent not inconsistent with the teachings herein.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skilled in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

1. A method of determining a presence, and/or aggressiveness level, and/or response to therapy of a human disease in a subject, said method comprising:
determining size of nanoparticles upon being exposed to a biological sample or a component of biological sample or pretreated biological sample from the subject to form an assay solution, wherein the average particle size of the assay solution is correlative to the presence, and/or aggressiveness level, and/or response to therapy of disease in said subject.

2. The method of claim 1, wherein said human disease is cancer.

3. The method of claim 1, wherein said nanoparticle is gold nanoparticle.

4. The method of claim 1, wherein said determining step is conducted via dynamic light scattering.

5. The method of claim 1, wherein said biological sample is untreated or pretreated with a chemical or biological substance before exposing to the said nanoparticles.

6. A method of determining a presence, and/or aggressiveness level, and/or response to therapy of a disease in a subject, said method comprising:
exposing a biological sample or a component of the biological sample or pretreated biological sample from the subject to a chemical or biological substance; and
analyzing a chemical interaction product produced upon said exposing.

7. The method of claim 6, where said chemical interaction product is an enzyme-substrate interaction product.

8. The method of claim 6, wherein said chemical or biological substance is an enzyme substrate, drug molecule, or immunoglobulin G (IgG).

9. The method of claim 7, wherein an enzyme involved in forming said enzyme-substrate interaction product is a protease

10. The method of claim 8, wherein said enzyme substrate is a peptide, carbohydrate, DNA, RNA, or a synthetic molecule, or a combination thereof.

11. The method of claim 6, wherein said chemical interaction product is a biomolecular complex.

12. (canceled)

13. The method of claim 6, wherein said analyzing is conducted by determining size of nanoparticles upon being exposed to a biological sample or a component of biological sample or pretreated biological sample from the subject to form an assay solution, wherein the average particle size of the assay solution is correlative to the presence, and/or aggressiveness level, and/or response to therapy of disease in said subject.

14. The method of claim 6, wherein said analyzing comprises utilization of mass spectroscopy, an immunoassay, UV-Vis absorption, fluorescence spectroscopy or microscopy, or chromatography, or magnetic property analysis.

15. The method of claim 6, wherein said chemical or biological substance is a drug molecule.

16. The method of claim 15, wherein the drug is Avastin.

17. (canceled)

18. A method for treatment of cancer, said method comprising administering an engineered immunoglobulin G (IgG) resistant to proteolytic breakdown in a subject, a composition comprising a matrix metalloprotease inhibitor followed by or simultaneous to anti-cancer drug co-administration, or human IgG from pooled human blood or its derivative product, or a combination thereof.

19-55. (canceled)

56. A method of detecting molecular interactions amongst biomolecules in a test sample, said method comprising combining a first biological sample with a second biological sample to form said test sample; and subjecting said test sample to an examination process configured to observe interactions with biomolecules of the first biological sample with biomolecules in the second biological sample.

57. (canceled)

58. The method of claim 56, wherein said first and second biological sample are from a common subject or from two different subjects.

59-65. (canceled)

66. The method of claim 13, wherein the cancer is prostate cancer.
